(19) United States
(12) Patent Application Publication
(10) Pub. No.: US 2025/0009646 A1
(43) Pub. Date: Jan. 9, 2025

(54) INJECTABLE SUSTAINED RELEASE PHARMACEUTICAL COMPOSITION

(71) Applicant: SpecGx, Webster Groves, MO (US)

(72) Inventors: Mark Worley, Webster Groves, MO (US); William Sanders, Webster Groves, MO (US)

(21) Appl. No.: 18/712,628

(22) PCT Filed: Nov. 22, 2022
(86) PCT No.: PCT/US2022/080349

§ 371 Date: May 22, 2024

Related U.S. Application Data
(60) Provisional application No. 63/282,014, filed on Nov. 22, 2021.

(51) Int. Cl. A61K 9/00; A61K 38/12

(52) U.S. Cl. CPC A61K 9/0019; A61K 9/0002; A61K 38/12

(57) ABSTRACT

Provided herein is a pre-filled injection syringe with an injectable sustained release pharmaceutical composition suitable for administration and methods of preparation thereof. The injectable sustained release pharmaceutical composition may be lanreotide acetate and have a density of 1.000 g/cm³ to 1.015 g/cm³.

### FIELD

This application relates to injectable sustained release pharmaceutical compositions and pre-filled injection syringes containing the same.

### BACKGROUND

Mixing of highly viscous pharmaceutical compositions containing peptides can often lead to a pharmaceutical composition with contamination, exposure to air, or degradation due to heating. Mixing of a viscous material can require a great deal of force to create shearing and even distribution of non-mixed materials. Hence, there is an ongoing need for improved homogeneously mixed injectable pharmaceutical compositions.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

Disclosed herein is a pre-filled injection syringe comprising an injectable sustained release pharmaceutical composition suitable for administration. The injectable sustained release pharmaceutical composition may include lanreotide acetate, and the injectable sustained release pharmaceutical composition has a density of 1.000 g/cm³ to 1.015 g/cm³.

Further disclosed herein is a method for preparing an injectable sustained release pharmaceutical composition. The method may include: mixing a composition comprising lanreotide acetate with water to form the injectable sustained release pharmaceutical composition; and filling an injection syringe with the injectable sustained release pharmaceutical composition. The injectable sustained release pharmaceutical composition has a density of 1.000 g/cm³ to 1.015 g/cm³.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional de-tails are not described in order to avoid obscuring the description.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

Articles “a” and “an” are used herein to refer to one or to more than one (i.e. at least one) of the grammatical object of the article. By way of example, “an element” means at least one element and can include more than one element.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the term “subject” and “patient” are used interchangeably herein and refer to both human and nonhuman animals.

Disclosed herein is an injectable sustained release pharmaceutical composition suitable for administration. In various aspects, the injectable sustained release pharmaceutical composition includes lanreotide acetate and has a density of 1.000 g/cm³ to 1.015 g/cm³. For example, the injectable sustained release pharmaceutical composition may have a density of 1.000 g/cm³ to 1.002 g/cm³, 1.001 g/cm³ to 1.003 g/cm³, 1.002 g/cm³ to 1.004 g/cm³, 1.003 g/cm³ to 1.005 g/cm³, 1.004 g/cm³ to 1.006 g/cm³, 1.005 g/cm³ to 1.007 g/cm³, 1.006 g/cm³ to 1.008 g/cm³, 1.007 g/cm³ to 1.009 g/cm³, 1.008 g/cm³ to 1.010 g/cm³, 1.009 g/cm³ to 1.011 g/cm³, 1.010 g/cm³ to 1.012 g/cm³, 1.011 g/cm³ to 1.013 g/cm³, 1.012 g/cm³ to 1.014 g/cm³, or 1.013 g/cm³ to 1.015 g/cm³.

In some embodiments, the injectable sustained release pharmaceutical composition is contained within a pre-filled injection syringe. The injection syringe is filled with a dose of 60 mg/0.2 mL, 90 mg/0.3 mL, or 120 mg/0.5 mL lanreotide acetate.

In some embodiments, the injectable sustained release pharmaceutical composition has few to substantially no bubbles distributed within the composition. In an aspect, the injectable sustained release pharmaceutical composition is a homogeneous semi-solid. Without being limited to any particular theory, the density of the composition may be related to the amount of air bubbles distributed in the composition. The injectable sustained release pharmaceutical composition is suitable for mixing without degradation. In some aspects, a homogeneously mixed composition with few to substantially no bubbles may provide for the injectable sustained release pharmaceutical composition to have a shelf life of at least 2 years within the injection syringe.

The injectable sustained release pharmaceutical composition is suitable for filling the injection syringe immediately after being mixed without requiring a phase change. For example, the injectable sustained release pharmaceutical composition is non-lyophilized and does not need to be reconstituted prior to administration.

In some aspects, the injectable sustained release pharmaceutical composition has a high viscosity. For example, the injectable sustained release pharmaceutical composition may have a viscosity of 3,000 milPoise to 10,000 milPoise, 20,000 milPoise to 40,000 milPoise, 30,000 milPoise to 50,000 milPoise, 40,000 milPoise to 60,000 milPoise, 50,000 milPoise to 70,000 milPoise, 60,000 milPoise to 80,000 milPoise, 70,000 milPoise to 90,000 milPoise, 80,000 milPoise to 100,000 milPoise, or up to 100,000 milPoise. The composition with a high viscosity of up to 100,000 milPoise is suitable for mixing to create a homogeneous mixture.

In an embodiment, the injection syringe may be stored in a foil pouch comprising an overlay of an inert gas to reduce impurity formation during sterilization. For example, the inert gas overlay may be a nitrogen overlay. The injection syringe may be stored in the foil pouch until the composition is ready to be injected into a patient. In some aspects, the foil pouch with the injection syringe may be sterilized by gamma irradiation. In some examples, the gamma irradiation may be at a dose ≥25 kGy. In other examples, the gamma irradiation may be at a dose of 20 kGy to 30 kGy. In some aspects, the nitrogen overlay further aids in the composition having a shelf life of at least 2 years.

Further disclosed herein is a method for preparing the injectable sustained release pharmaceutical composition. In some embodiments, the method may include mixing a composition comprising a somatostatin analog salt and an aqueous acid solution with water to form the injectable sustained release pharmaceutical composition and filling an injection syringe with the injectable sustained release pharmaceutical composition.

In an embodiment, the injectable sustained release pharmaceutical composition has a density of 1.000 g/cm³ to 1.015 g/cm³. For example, the injectable sustained release pharmaceutical composition may have a density of 1.000 g/cm³ to 1.002 g/cm³, 1.001 g/cm³ to 1.003 g/cm³, 1.002 g/cm³ to 1.004 g/cm³, 1.003 g/cm³ to 1.005 g/cm³, 1.004 g/cm³ to 1.006 g/cm³, 1.005 g/cm³ to 1.007 g/cm³, 1.006 g/cm³ to 1.008 g/cm³, 1.007 g/cm³ to 1.009 g/cm³, 1.008 g/cm³ to 1.010 g/cm³, 1.009 g/cm³ to 1.011 g/cm³, 1.010 g/cm³ to 1.012 g/cm³, 1.011 g/cm³ to 1.013 g/cm³, 1.012 g/cm³ to 1.014 g/cm³, or 1.013 g/cm³ to 1.015 g/cm³. In some examples, the density of the injectable sustained release pharmaceutical composition may be about 0.004 higher prior to sterilization. In other examples, the density of the injectable sustained release pharmaceutical composition may be 0.004, 0.005, 0.006, 0.007, 0.008, 0.009, or 0.010 g/cm³ lower after sterilization.

The somatostatin analog is lanreotide. The acid is acetic acid. The injectable sustained release pharmaceutical composition comprises lanreotide acetate. The injection syringe is filled with a dose of 60 mg/0.2 mL, 90 mg/0.3 mL, or 120 mg/0.5 mL lanreotide acetate.

In some embodiments, the mixing may be performed in a static mixer. In an example, the static mixer may be a variable speed static vacuum mixer. The static mixer may include an orifice plate located between two opposing pistons within a collection cylinder, such that the pistons are operable to push the composition through the orifice plate, thereby mixing the composition.

In some embodiments, the mixing step further includes pushing the injectable sustained release pharmaceutical composition through a plurality of orifices on the orifice plate in the static mixer. The injectable sustained release pharmaceutical composition is pushed through the orifice plate using the pair of opposing pistons in the static mixer. In an embodiment, the plurality of orifices are angled. The orifices may be cut in offset angels to cause the material to swirl at it extrudes out the orifices. In some examples, the angle of the orifices may vary across the orifice plate. In additional embodiments, the plurality of orifices are arranged in concentric circles on the orifice plate and the distance between orifices in a first concentric circle is different from the distance between orifices in a second concentric circle. The angle of the orifices in each row (e.g. concentric circle) may be slightly different to create a slightly different flow direction, allowing for more interaction between semi solid material as the streams exit the orifices. In at least one example, the orifice plate has 5 holes in the middle that are cut at 90 degrees off the bottom plan. The successive rows from the middle may be 76 degrees, 104 degrees, 119 degrees, and 120 degrees off of the bottom plan, respectively.

The orifices in the orifice plate may be operable to create shearing as the composition enters the orifice plate. For example, the orifices may act as an extrusion device forcing the viscous composition to extrude in tube like structures as it exits the orifice plate. The angle of the orifices may force the material from one extrusion stream to collide with another, thereby folding a first mixture of the composition onto a new mixture of the composition. Because of the orifice positions in the plate, the extrusion force drives the viscous composition to a new location on the plate resulting in the composition being located in a new section of the collection cylinder. As the piston drives the composition back through the orifice plate, the composition enters a new set of orifices which may result in shearing and collision with a new mixture of the composition. The repetitive movement of the composition through the orifice plate may result in homogeneous mixing without substantive production of bubbles.

In some embodiments, the mixing step further comprises removing atmospheric gasses from the static mixer and applying a vacuum. A vacuum system can create a vacuum of 0.08 TorrA within the cylinder of the mixer. The force upon the material can cause counterforces so the mixer may withstand pressures up to 1000 PSI.

In an embodiment, the injectable sustained release pharmaceutical composition is not degraded during mixing. For example, the mixer allows mixing at ambient temperature. This is an advantage as very viscous material sometimes has to be heated to allow mixing or the containment vessel must be cooled because of friction created by the mixing process, both of which can affect the composition being mixed and potentially cause degradation of proteins or other components of the composition.

In some aspects, the injectable sustained release pharmaceutical composition has a high viscosity and is suitable for mixing. For example, the injectable sustained release pharmaceutical composition may have a viscosity of 3,000 milPoise to 10,000 milPoise, 20,000 milPoise to 40,000 milPoise, 30,000 milPoise to 50,000 milPoise, 40,000 milPoise to 60,000 milPoise, 50,000 milPoise to 70,000 milPoise, 60,000 milPoise to 80,000 milPoise, 70,000 milPoise to 90,000 milPoise, 80,000 milPoise to 100,000 milPoise, or up to 100,000 milPoise. The composition with a high viscosity of up to 100,000 milPoise is suitable for mixing to create a homogeneous mixture.

In an embodiment, the method may further include replacing the orifice plate with a transfer plate to fill the injection syringe. In another embodiment, the orifice plate is removed and replaced with a single nozzle funnel for material transfer. The cylinder and piston may then become a transfer vessel, greatly reducing product loss compared to if a separate transfer vessel was used.

In some embodiments, the injectable sustained release pharmaceutical composition has few to substantially no bubbles distributed within the composition. In an aspect, the injectable sustained release pharmaceutical composition is a homogeneous mixture. Without being limited to any particular theory, the density of the composition may be related to the amount of air bubbles distributed in the composition. The injectable sustained release pharmaceutical composition is suitable for mixing without degradation. In some aspects, a homogeneously mixed composition with few to substantially no bubbles may provide for the injectable sustained release pharmaceutical composition to have a shelf life of at least 2 years within the injection syringe.

The injectable sustained release pharmaceutical composition is suitable for filling the injection syringe immediately after being mixed without requiring a phase change. For example, the injectable sustained release pharmaceutical composition is non-lyophilized and does not need to be reconstituted prior to administration. The injectable sustained release pharmaceutical composition is suitable for administration immediately after mixing.

In an embodiment, the method may further include storing the filled injector syringe in a foil pouch comprising an inert gas to reduce impurity formation during sterilization. In some examples, the inert gas is nitrogen and a nitrogen overlay is added to the pouch. In some aspects, the foil pouch with the injection syringe may be sterilized by gamma irradiation. In some examples, the gamma irradiation may be at a dose ≥25 kGy. In other examples, the gamma irradiation may be at a dose of 20 kGy to 30 kGy.

In one embodiment, any sterilization technique such as gamma irradiation, electron beam irradiation, steam or sterile filtration for part of the process may be used to obtain a sterile pharmaceutical composition, wherein the composition comprises an injectable sustained release pharmaceutical composition comprising lanreotide acetate, and wherein the injectable sustained release pharmaceutical composition has a density of 1.000 g/cm³ to 1.015 g/cm³. Gamma sterilization may be effective to attain a 10-6 probability of microbial survival without excessive heating of the composition or exposure to toxic chemicals, without any loss of active pharmaceutical ingredient potency of the injectable sustained release pharmaceutical composition comprising lanreotide acetate.

### Exemplary Embodiments

Embodiment 1: A pre-filled injection syringe comprising an injectable sustained release pharmaceutical composition suitable for administration, the injectable sustained release pharmaceutical composition comprising lanreotide acetate, wherein the injectable sustained release pharmaceutical composition has a density of 1.000 g/cm³ to 1.015 g/cm³.

Embodiment 2: The pre-filled injection syringe of embodiment 1, wherein the injection syringe is filled with a dose of 60 mg/0.2 mL, 90 mg/0.3 mL, or 120 mg/0.5 mL lanreotide acetate.

Embodiment 3: The pre-filled injection syringe of embodiment 1, wherein the injectable sustained release pharmaceutical composition has substantially no bubbles.

Embodiment 4: The pre-filled injection syringe of embodiment 1, wherein the injectable sustained release pharmaceutical composition is a homogeneous mixture.

Embodiment 5: The pre-filled injection syringe of embodiment 1, wherein the injectable sustained release pharmaceutical composition is suitable for filling the injection syringe immediately after being mixed without requiring a phase change.

Embodiment 6: The pre-filled injection syringe of embodiment 1, wherein the injectable sustained release pharmaceutical composition within the injection syringe has a shelf life of at least 2 years.

Embodiment 7: The pre-filled injection syringe of embodiment 1, wherein the injectable sustained release pharmaceutical composition is non-lyophilized and does not need to be reconstituted prior to administration.

Embodiment 8: The pre-filled injection syringe of embodiment 1, wherein the injectable sustained release pharmaceutical composition is suitable for mixing without degradation.

Embodiment 9: The pre-filled injection syringe of embodiment 1, wherein the injectable sustained release pharmaceutical composition is suitable for mixing at a viscosity of up to 100,000 milPoise.

Embodiment 10: The pre-filled injection syringe of embodiment 1, wherein the injection syringe is stored in a foil pouch comprising a nitrogen overlay.

Embodiment 11: A non-lyophilized injectable sustained release pharmaceutical composition suitable for administration, the injectable sustained release pharmaceutical composition comprising lanreotide acetate, wherein the injectable sustained release pharmaceutical composition has a density of 1.000 g/cm³ to 1.015 g/cm³.

Embodiment 12: The pharmaceutical composition of embodiment 11, wherein injectable sustained release pharmaceutical composition is contained in an injection syringe at a dose of 60 mg/0.2 mL, 90 mg/0.3 mL, or 120 mg/0.5 mL lanreotide acetate.

Embodiment 13: The pharmaceutical composition of embodiment 11, wherein the injectable sustained release pharmaceutical composition has substantially no bubbles.

Embodiment 14: The pharmaceutical composition of embodiment 11, wherein the injectable sustained release pharmaceutical composition is a homogeneous mixture.

Embodiment 15: The pharmaceutical composition of embodiment 11, wherein the injectable sustained release pharmaceutical composition is suitable for filling a syringe immediately after mixing without requiring a phase change.

Embodiment 16: The pharmaceutical composition of embodiment 11, wherein the injectable sustained release pharmaceutical composition is filled within an injection syringe and has a shelf life of at least 2 years.

Embodiment 17: The pharmaceutical composition of embodiment 16, wherein the injection syringe is stored in a foil pouch comprising a nitrogen overlay.

Embodiment 18: The pharmaceutical composition of embodiment 11, wherein the injectable sustained release pharmaceutical composition does not need to be reconstituted prior to administration.

Embodiment 19: The pharmaceutical composition of embodiment 11, wherein the injectable sustained release pharmaceutical composition is suitable for mixing without degradation.

Embodiment 20: The pharmaceutical composition of embodiment 11, wherein the injectable sustained release pharmaceutical composition is suitable for mixing at a viscosity of up to 100,000 milPoise.

Embodiment 21: A method for preparing an injectable sustained release pharmaceutical composition, the method comprising: mixing a composition comprising a somatostatin analog salt and an aqueous acid solution with water to form the injectable sustained release pharmaceutical composition; and filling an injection syringe with the injectable sustained release pharmaceutical composition, wherein the injectable sustained release pharmaceutical composition has a density of 1.000 g/cm³ to 1.015 g/cm³.

Embodiment 22: The method of embodiment 21, wherein the somatostatin analog is lanreotide.

Embodiment 23: The method of embodiment 21, wherein the acid is acetic acid.

Embodiment 24: The method of embodiment 21, wherein the injectable sustained release pharmaceutical composition comprises lanreotide acetate.

Embodiment 25: The method of embodiment 24, wherein the injection syringe is filled with a dose of 60 mg/0.2 mL, 90 mg/0.3 mL, or 120 mg/0.5 mL lanreotide acetate.

Embodiment 26: The method of embodiment 21, wherein the mixing step further comprises pushing the injectable sustained release pharmaceutical composition through a plurality of orifices on an orifice plate in a static mixer.

Embodiment 27: The method of embodiment 26, wherein the injectable sustained release pharmaceutical composition is pushed through the orifice plate using a pair of opposing pistons in the static mixer.

Embodiment 28: The method of embodiment 26, wherein the plurality of orifices are angled.

Embodiment 29: The method of embodiment 26, wherein the plurality of orifices are arranged in concentric circles on the orifice plate and the distance between orifices in a first concentric circle is different from the distance between orifices in a second concentric circle.

Embodiment 30: The method of embodiment 26, wherein the mixing step further comprises removing atmospheric gasses from the static mixer and applying a vacuum.

Embodiment 31: The method of embodiment 26, wherein the injectable sustained release pharmaceutical composition is suitable for mixing at a viscosity of up to 100,000 milPoise.

Embodiment 32: The method of embodiment 26, further comprising replacing the orifice plate with a transfer plate to fill the injection syringe.

Embodiment 33: The method of embodiment 21, wherein the injectable sustained release pharmaceutical composition not degraded during mixing.

Embodiment 34: The method of embodiment 21, wherein the injectable sustained release pharmaceutical composition has substantially no bubbles distributed within the composition.

Embodiment 35: The method of embodiment 21, wherein the injectable sustained release pharmaceutical composition is a homogeneous mixture.

Embodiment 36: The method of embodiment 21, wherein the injectable sustained release pharmaceutical composition within the injection syringe has a shelf life of at least 2 years.

Embodiment 37: The method of embodiment 21, wherein the injectable sustained release pharmaceutical composition is not lyophilized and does not need to be reconstituted prior to administration.

Embodiment 38: The method of embodiment 21, wherein the injectable sustained release pharmaceutical composition is suitable for administration immediately after mixing.

Embodiment 39: The method of embodiment 21, wherein the injectable sustained release pharmaceutical composition is suitable for filling the injection syringe immediately after mixing without requiring a phase change.

Embodiment 40: The method of embodiment 21, further comprising storing the filled injector syringe in a foil pouch comprising a nitrogen overlay.

1. A pre-filled injection syringe comprising an injectable sustained release pharmaceutical composition suitable for administration, the injectable sustained release pharmaceutical composition comprising lanreotide acetate, wherein the injectable sustained release pharmaceutical composition has a density of 1.000 g/cm³ to 1.015 g/cm³.

2. (canceled)

3. The pre-filled injection syringe of claim 1, wherein the injectable sustained release pharmaceutical composition is a homogeneous mixture and has substantially no bubbles.

4. (canceled)

5. The pre-filled injection syringe of claim 1, wherein the injectable sustained release pharmaceutical composition is suitable for filling the injection syringe immediately after being mixed without requiring a phase change, is non-lyophilized, and does not need to be reconstituted prior to administration.

6. The pre-filled injection syringe of claim 1, wherein the injectable sustained release pharmaceutical composition within the injection syringe has a shelf life of at least 2 years.

7. (canceled)

8. The pre-filled injection syringe of claim 1, wherein the injectable sustained release pharmaceutical composition is suitable for mixing without degradation and at a viscosity of up to 100,000 milPoise.

9. (canceled)

10. The pre-filled injection syringe of claim 1, wherein the injection syringe is stored in a foil pouch comprising a nitrogen overlay.

11. A non-lyophilized injectable sustained release pharmaceutical composition suitable for administration, the injectable sustained release pharmaceutical composition comprising lanreotide acetate, wherein the injectable sustained release pharmaceutical composition has a density of 1.000 g/cm³ to 1.015 g/cm³.

12. (canceled)

13. The pharmaceutical composition of claim 11, wherein the injectable sustained release pharmaceutical composition is a homogeneous mixture and has substantially no bubbles.

14. (canceled)

15. The pharmaceutical composition of claim 11, wherein the injectable sustained release pharmaceutical composition is suitable for filling a syringe immediately after mixing without requiring a phase change and does not need to be reconstituted prior to administration.

16. The pharmaceutical composition of claim 11, wherein the injectable sustained release pharmaceutical composition is filled within an injection syringe and has a shelf life of at least 2 years.

17. The pharmaceutical composition of claim 16, wherein the injection syringe is stored in a foil pouch comprising a nitrogen overlay.

18. (canceled)

19. The pharmaceutical composition of claim 11, wherein the injectable sustained release pharmaceutical composition is suitable for mixing without degradation and at a viscosity of up to 100,000 milPoise.

20. (canceled)

21. A method for preparing an injectable sustained release pharmaceutical composition, the method comprising:
mixing a composition comprising a somatostatin analog salt and an aqueous acid solution with water to form the injectable sustained release pharmaceutical composition; and
filling an injection syringe with the injectable sustained release pharmaceutical composition,
wherein the injectable sustained release pharmaceutical composition has a density of 1.000 g/cm³ to 1.015 g/cm³.

22. The method of claim 21, wherein the somatostatin analog is lanreotide and the acid is acetic acid.

23. (canceled)

24. The method of claim 21, wherein the injectable sustained release pharmaceutical composition comprises lanreotide acetate.

25. (canceled)

26. The method of claim 21, wherein the mixing step further comprises pushing the injectable sustained release pharmaceutical composition through a plurality of orifices on an orifice plate in a static mixer using a pair of opposing pistons in the static mixer.

27. (canceled)

28. The method of claim 26, wherein the plurality of orifices are angled.

29. The method of claim 26, wherein the plurality of orifices are arranged in concentric circles on the orifice plate and the distance between orifices in a first concentric circle is different from the distance between orifices in a second concentric circle.

30. The method of claim 26, wherein the mixing step further comprises removing atmospheric gasses from the static mixer and applying a vacuum.

31. (canceled)

32. The method of claim 26, further comprising replacing the orifice plate with a transfer plate to fill the injection syringe.

33. The method of claim 21, wherein the injectable sustained release pharmaceutical composition not degraded during mixing and is suitable for mixing at a viscosity of up to 100,000 milPoise.

34. The method of claim 21, wherein the injectable sustained release pharmaceutical composition is a homogeneous mixture and has substantially no bubbles distributed within the composition.

35. (canceled)

36. The method of claim 21, wherein the injectable sustained release pharmaceutical composition within the injection syringe has a shelf life of at least 2 years.

37. The method of claim 21, wherein the injectable sustained release pharmaceutical composition is not lyophilized, and does not need to be reconstituted prior to administration, and is suitable for administration immediately after mixing.

38. (canceled)

39. (canceled)

40. The method of claim 21, further comprising storing the filled injector syringe in a foil pouch comprising a nitrogen overlay.
